(19) United States
(12) Patent Application Publication
(10) Pub. No.: US 2005/0288258 A1
(43) Pub. Date: Dec. 29, 2005

(54) Compounds, compositions and methods for treating or preventing viral infections and associated diseases

(76) Inventors: Guy D. Diana, Pottstown, PA (US); Thomas R. Bailey, Phoenixville, PA (US); Dorothy C. Young, Gainesville, VA (US); Srinivas K Chunduru, West Chester, PA (US)

Correspondence Address: DANN, DORFMAN, HERRELL & SKILLMAN, 1601 MARKET STREET, SUITE 2400, PHILADELPHIA, PA, 19103-2307, US

(21) Appl. No.: 10/511,430

(22) PCT Filed: Apr. 23, 2003
(86) PCT No.: PCT/US03/12192

§ 371 Date: Aug. 18, 2005

Related U.S. Application Data
(60) Provisional application No. 60/374,740, filed on Apr. 23, 2002.

(51) Int. Cl. A61K 31/675

(52) U.S. Cl. 514/89; 546/22

(57) ABSTRACT

Pyridine derivatives that exhibit antiviral activity are disclosed.

### FIELD OF THE INVENTION

The present invention relates to novel pyridine derivatives, as well as compositions containing such derivatives and their use for treating or preventing viral infections and diseases associated with such infections, particularly those viral infections and associated diseases caused by viruses within the Flaviviridae family.

### BACKGROUND OF THE INVENTION

The Flaviviridae family consists of three genera and several viruses that are currently unassigned to specific genera. The hepacivirus genus includes the hepatitis C viruses (HCV). Viruses such as GB virus-A and GB virus-A-like agents, GB virus-B and GBV-C or hepatitis G virus, while at present not formally classified within the hepacivirus genus, are closely related to HCV and represent unassigned members of the Flaviviridae family. Also within the Flaviviridae is the pestivirus genus, which includes bovine viral diarrhea viruses (BVDV), border disease viruses and classical swine fever virus, and the flavivirus genus, with viruses such as dengue, yellow fever, Japanese encephalitis and tick-borne encephalitis viruses.

Viruses within this family cause significant disease in human and animal populations. HCV is a major cause of human hepatitis globally. The World Health Organization estimates that 170 million people worldwide are presently infected with the virus. Most infections become persistent and about 60% of cases develop chronic liver disease. Chronic HCV infection can lead to development of cirrhosis, hepatocellular carcinoma and liver failure.

Interferon and interferon in combination with ribavirin are used in the U.S. to treat hepatitis caused by HCV. These treatments are associated with improved serum enzyme response in some patients. The remainder are non-responsive to treatment. For responders, a sustained clinical improvement is seen in only a small percentage of patients; the majority of patients relapse upon cessation of treatment. Thus, the effectiveness of therapy for chronic hepatitis C is variable and its cure rate remains low. Moreover, therapy is often associated with considerable side effects.

Pestivirus infections of domesticated livestock cause significant economic losses worldwide. Pestiviruses cause a range of clinical manifestations including abortion, teratogenesis, respiratory problems, chronic wasting disease, immune system dysfunction and predisposition to secondary viral and bacterial infections. Certain BVDV strains cause an acute fatal disease. BVDV can also establish persistent infections in fetuses. When born, these persistently infected (PI) animals remain viremic throughout life and serve as continuous virus reservoirs. PI animals often succumb to fatal mucosal disease.

Flaviviruses are important pathogens of man and are also prevalent throughout the world. There are at least 38 flaviviruses associated with human disease, including the dengue fever viruses, yellow fever virus and Japanese encephalititis virus. Flaviviruses cause a range of acute febrile illnesses and encephalitic and hemorrhagic diseases.

Currently, there are no antiviral pharmaceuticals to prevent or treat pestivirus or flavivirus infections.

New therapies and preventatives are clearly needed for infections and diseases caused by viruses of the Flaviviridae family.

In considering approaches to the diagnosis, control, prevention and treatment of infections and associated diseases caused by viruses, it is often desirable to identify virus-specific functions that may be exploited in the implementation of such approaches. In particular, enzymatic activities of virus-encoded polypeptides are quite useful. These virus-specified components are often essential for virus replication and may be suitable targets for antiviral drug discovery strategies.

One such target that plays a central role in the life cycle of many RNA viruses is the virus-encoded RNA-dependent RNA polymerase (RdRp) protein. Regarding viruses of the Flaviviridae, this protein is termed NS5B in the case of the hepaciviruses and pestiviruses, and NS5 in the case of the flaviviruses (collectively referred to as NS5). RdRp proteins are a key component of the virus replicase complex, enabling the virus to replicate its RNA genome and produce progeny viruses. The RdRp of RNA viruses is an attractive target for antiviral drug development.

### SUMMARY OF THE INVENTION

In accordance with one aspect, the present invention provides compounds for preventing or treating viral infections and for preventing or treating diseases associated with viral infections in living hosts. The compounds of the invention have the following general structure: 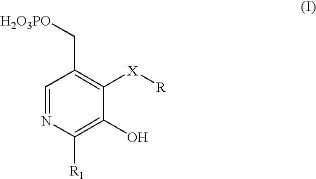
wherein X represents a divalent linking moiety selected from the group consisting of —CH═N— and —CH═CRₐ—;
R represents a radical selected from the group consisting of an unsubstituted or substituted alkyl (C₁-C₆) radical, an unsubstituted or substituted aryl (C₆-C₁₄) radical, an unsubstituted or substituted aralkyl (C₇-C₁₅) radical, an unsubstituted or substituted heterocyclic radical, or a radical of the formula —NRₐ—X′—R_{b}, wherein X′ represents a valence bond or a divalent linking moiety selected from the group of —C(═O)—, —S(═O)₂— or —(CH₂)ₙ—, n being an integer from 1 to 6;
Rₐ represents hydrogen or an unsubstituted or substituted alkyl (C₁-C₆) radical; Rb represents hydrogen, an unsubstituted or substituted alkyl (C₁-C₆) radical, an unsubstituted or substituted aryl (C₆-C₁₄) radical, an unsubstituted or substituted aralkyl (C₇-C₁₆) radical, an unsubstituted or substituted heterocyclic radical, an unsubstituted or substituted alicyclic (C₅-C₇) radical or a carbalkoxy radical;
R₁ represents an unsubstituted or substituted alkyl (C₁-C₆) radical;
the heterocyclic radical represented by R or R_{b} being at least one selected from the group of furan, thiophene, pyrrole, tetrazole, pyridine, piperidine, morpholine, pyrazole, pyridazine, triazole, pyrimidine, oxadiazole, thiadiazole, oxazole, isoxazole, isothiazole or azepane; the alkyl radical substituent(s) being at least one selected from the group of carboxy, hydroxy, alkoxy, amino, alkylamino, dialkylamino, thiol or alkylthio; the aryl radical substituent(s) and the aralkyl radical substituent(s) being at least one selected from the group of a straight or branched chain, saturated or unsaturated aliphatic group having 1-6 carbon atoms, halogen, nitro, carboxy, hydroxy, hydroxyalkyl, perhaloalkyl, monohaloalkyl, dihaloalkyl, alkoxy, perhaloalkoxy, phenylalkoxy, acyl, acyloxy, acyloxyalkyl, cyano, carbalkoxy, thiol, alkylthio, alkylsulfinyl, alkylsulfonyl, amino, alkylamino, dialkylamino, aminoalkyl, alkylaminoalkyl, dialkylaminoalkyl, sulfonamido, carboxamido or alkanoylamino; the heterocyclic radical substituent(s) and the alicyclic radical substituents(s) being at least one selected from the group of a straight or branched chain, saturated or unsaturated aliphatic group having 1-6 carbon atoms, halogen, perhaloalkyl, monohaloalkyl, dihaloalkyl, alkoxy, acyl, acyloxy, acyloxyalkyl, phenylalkoxy, hydroxy, hydroxyalkyl, alkylsulfonate, thiol, alkylthio, alkylsulfinyl, alkylsulfonyl, nitro, carboxy, carbalkoxy, or an unsubstituted or substituted aryl (C₆-C₁₄) radical; the isomeric forms and the pharmaceutically acceptable salts of the above compound.

According to another aspect, the present invention provides pharmaceutical compositions comprising one or more of the above-described pyridine derivatives in combination with a pharmaceutically acceptable carrier medium.

In accordance with yet another aspect, the present invention provides methods for the treatment or prophylaxis of viral infections in living hosts by administering an effective amount of at least one compound of the invention to a host that is susceptible to, or suffering from such infection.

### DETAILED DESCRIPTION OF THE INVENTION

Pyridine derivatives according to the present invention can be conveniently prepared from known starting materials or from intermediates that may be prepared from known starting materials using conventional chemistry knowledge and skills, e.g. by following one of the general synthetic schemes shown below, wherein R, Rₐ and R¹ are as previously defined:

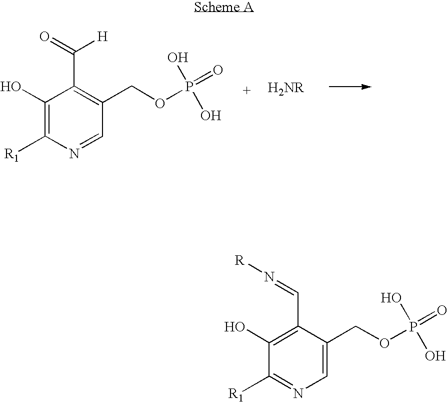

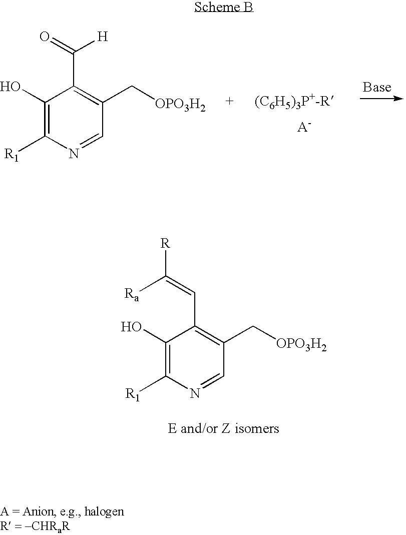

In general synthetic scheme A, pyridoxal-5′-phosphate monohydrate is reacted with an amine reactant appropriate to yield the desired “R” substituent in the compound of formula (I), above. The reaction is conveniently conducted in a reaction medium, such as ethanol, heated to reflux for one to twelve hours.

General synthetic scheme B involves the reaction of pyridoxal phosphate with the appropriate triphenylphosphonium halide in the presence of n-butyl lithium or another appropriate base, such as a different carbanion, an alkoxide, or a metal hydride.

In the products obtained from the above-described reactions, the divalent linking moiety (X) is bound to the aromatic ring through a methine group.

Preparation of specific antiviral compounds which may be used in the practice of this invention are exemplified below. Starting materials for carrying out these reactions are available from commercial sources or are intermediates that may be prepared from known starting materials using conventional chemistry knowledge and skills.

In vitro studies have been performed which demonstrate the usefulness of compounds described herein as antiviral agents. Antiviral activity was measured by the inhibitory activity of the compounds against the viral RdRp in an enzymological assay for RNA synthesis.

Compounds having particular utility, including isomeric forms, are the pyridine derivatives shown in formula (I), above, in which X—R represents —C═N-aryl; —C═N-het; —C═N—NH—S (═O)₂-aryl; —C═N—NH—S (═O)₂-het; —C═N—NH—C (═O)-aryl; —C═N—NH—C (═O)-het; —C═N—NH—C(═O)-alkyl; —C═N—NH—C(═O)—H; —C═N—NH-aryl; —C═N—NH-aralkyl; —C═N—NH-het; —C═N—NH-cycloalkyl; and —C═N—NH— (CH₂)ₙ—C (═O)—O-alkyl (n=1−6), wherein “het” represents a heterocyclic radical, as previously defined with reference to formula I, above, and the heterocyclic radicals, the aryl radicals, the aralkyl radicals, the alkyl radicals, and the cycloalkyl radicals in this group of compounds may be unsubstituted or substituted, the heterocyclic radical substituent(s), the aryl radical substituent(s), the aralkyl radical substituent(s), and the alkyl radical substituents being as previously set forth with reference to formula I, above. The cycloalkyl radical substituents may be the same as the alicyclic radical substituents specified with reference to formula I, above.

Compounds of the invention also include those having the formula: 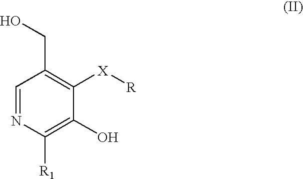
wherein, each of X, R₁, and R is as defined above, the isomeric forms of said compound and the pharmaceutically acceptable salts of said compound.

Compounds of formula II may be prepared by dephosphorylation of the corresponding compound of formula I, using methods and knowledge familiar to those of skill in the art. As previously noted, the compounds of formula I can be prepared from known starting materials, or from intermediates that can be prepared from known starting materials using conventional chemistry knowledge and skills. Alternatively, compounds of formula II may be synthesized according to Schemes C and D.

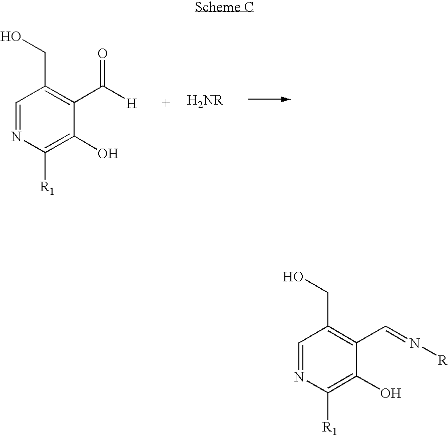

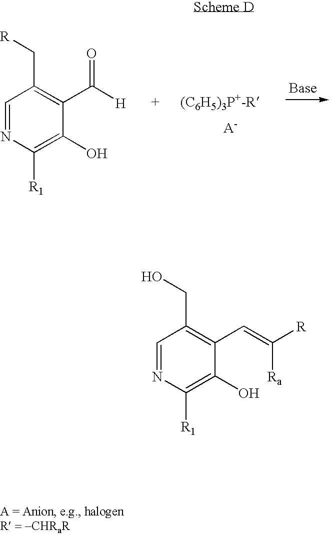

In schemes C and D, R, Rₐ and R₁ are as defined above. Scheme D illustrates a Wittig-type reaction analogous to that depicted in scheme B; therefore, the bases that are appropriate in the reaction of scheme B will also be appropriate in that of scheme D.

The compounds of the invention may be administered as such, or in a form from which the active agent can be derived, such as a prodrug. A prodrug is a derivative of a compound described herein, the pharmacologic action of which results from the conversion by chemical or metabolic processes in vivo to the active compound. Prodrugs include, without limitation, ester derivatives of the compounds of formula I, above. Carboxylate esters may be prepared, for example, by reacting simple or functionalized carboxylic acids with the hydroxyl group of the pyridine derivative. Other prodrugs may be prepared according to procedures well known in the field of medicinal chemistry and pharmaceutical formulation science. See, e.g., References 1 and 2, below.

Prodrugs, in accordance with the present invention, include, without limitation, compounds having the formula: 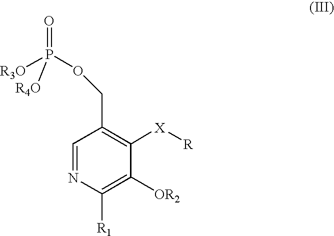
wherein X, R, and R₁ are as previously defined, and R₂, R₃ and R₄ may be the same or different, and represent hydrogen or a radical selected from the group consisting of substituted or unsubstituted, straight or branched chain alkyl (C₁-C₆), substituted or unsubstituted alicyclic (C₅-C₇), substituted or unsubstituted aryl (C₆-C₁₄) radicals, an acyl radical, an acyloxyalkyl radical or an amino acid residue (—C(═O)—CHR_{c}—NH₂, wherein R_{c} is the side chain of a naturally occurring amino acid), the isomeric forms of said compound and the pharmaceutically acceptable salts of said compound.

The substituted alkyl, alicyclic and aryl radicals represented by R₂, R₃, and R₄ are the same as previously described, with reference to the compounds of Formula I.

Prodrugs of formula III may be synthesized from known starting materials or from intermediates that can be prepared from known starting materials using conventional chemistry knowledge and skills. See also the References listed below.

Although compounds of formula III may serve as prodrugs in accordance with this invention, their utility is not so limited. For example, compounds of formula III, per se, exhibit pharmaceutical activity. Furthermore, compounds of formula III may also serve as intermediates in the synthesis of compounds of formula I by reactions that are familiar to those of skill in the art.

The term “aryl” as used herein refers to a carbocyclic, aromatic radical of six to fourteen carbon atoms and includes, without limitation, phenyl, naphthyl, fluorenyl, anthracenyl, indanyl or the like.

The term “alkyl” as used herein refers to aliphatic hydrocarbon radicals of one to six carbon atoms in length. Similarly, the term “alkyl”, or any variation thereof, used in combination form to name substituents, such as aralkyl, phenylalkyl, alkoxy, alkylthio, alkylamino, alkylsulfinyl or alkylsulfonyl also refers to aliphatic hydrocarbon radicals of one to six carbon atoms in length.

The term “acyl”, or any variation thereof, used in combination form to name substituents, e.g. acyloxyalkyl, refers to a radical which is derived from a carboxylic acid by the removal of the hydroxyl groups, and which may be either aliphatic (straight or branched chain C₁-C₆) or aromatic (C₆-C₁₄).

The term “carboxamido”, as used herein, refers to a radical or substituent of the formula —C(═O)—NR″R′″, wherein R″ and R′″ represent hydrogen or alkyl (C₁-C₆).

The term “sulfonamido”, as used herein, refers to a radical or substituent of the formula —SO₂—NR″R′″ or —NR″—SO₂R′″, wherein R″ and R′″ are as previously defined.

The term “alkanoylamino”, as used herein, refers to a radical or substituent of the formula —NH—C(═O)—R″, wherein R″ is as previously defined.

The term “carbalkoxy”, as used herein, refers to a radical or substituent —C (═O)—OR″, wherein R″ is as previously defined.

The compounds of formula I, II, and III, above, their isomers and their pharmaceutically acceptable salts exhibit antiviral activity. The compounds of the invention are particularly effective against viruses of the Flaviviridae family and are useful in the treatment and prophylaxis of infections and diseases associated with these viruses in living hosts.

The compounds of the present invention or precursors thereof and their isomeric forms and pharmaceutically acceptable salts thereof are also useful in treating and preventing viral infections, in particular hepatitis C infection, and diseases in living hosts when used in combination with each other (i.e. pharmaceutical compositions comprising the compounds are administered concurrently with each other or sequentially, in either order). The combination of compounds provided herein may further be provided to a subject in respective pharmaceutical compositions, concurrently with or sequentially to other biologically active agents, including but not limited to the group consisting of interferon, a pegylated interferon, ribavirin, protease inhibitors, polymerase inhibitors, small interfering RNA compounds, anti-sense compounds, nucleotide analogs, nucleoside analogs, immunoglobulins, immunomodulators, hepatoprotectants, anti-inflammatory agents, antibiotics, antivirals, and anti-infective compounds. The present invention further provides combination therapy in which two or more pyridine derivatives, i.e., at least two pharmaceutical compositions, each comprising a different compound of the present invention, are provided to a subject in need thereof either concurrently with each other or sequentially, and such therapy may further comprise providing concurrently or sequentially other therapeutic agents or potentiators, such as acyclovir, famicyclovir, valgancyclovir and related compounds, ribavirin and related compounds, amantadine and related compounds, various interferons such as, for example, interferon-alpha, interferon-beta, interferon-gamma and the like, as well as alternative forms of interferons such as pegylated interferons. Additionally, combinations of, for example ribavirin and interferon, may be administered as an additional combination for a multiple combination therapy with at least one of the compounds of the present invention.

The combination therapy with any of the above-described biologically active agents may also be sequential, that is the treatment with a first pharmaceutical composition comprising a compound of the invention followed by treatment with a second pharmaceutical composition comprising a second compound of the invention, wherein the second compound is different than the first compound. Alternatively, treatment may be with both two or more pharmaceutical compositions, wherein each pharmaceutical composition comprises a different compound of the invention, at the same time. The sequential therapy can be within a reasonable time after the completion of the first therapy with the pharmaceutical composition. Treatment with the respective pharmaceutical compositions, each comprising a different compound of the present invention, at the same time may be provided in the same daily dose or in separate doses. Combination therapy may also be provided wherein a pharmaceutical composition comprising at least one compound of the present invention is administered in a composition further comprising at least one biologically active agent, i.e. in a single dose. The dosages for both concurrent and sequential combination therapy (for combined pharmaceutical compositions comprising at least two compounds of the invention or compositions comprising at least one compound of the invention and at least one biologically active agent), will depend on absorption, distribution, metabolism and excretion rates of the components of the pharmaceutical composition as well as other factors known to one of skill in the art. Dosage values of the pharmaceutical composition will also vary with the severity of the condition to be alleviated. It is to be further understood that for any particular subject, specific dosage regimens and schedules may be adjusted over time according to the individual's need and the professional judgment of the person administering or supervising the administration of the pharmaceutical compositions.

In a further embodiment, the compounds of the invention may be used for the treatment of HCV in humans in combination therapy mode with other inhibitors of the HCV polymerase.

In yet a further embodiment, the compounds of the present invention may be used for the treatment of HCV in humans in combination therapy mode with other inhibitors of the HCV life cycle such as, for example, inhibitors of HCV cell attachment or virus entry, HCV translation, HCV RNA transcription or replication, HCV maturation, assembly or virus release, or inhibitors of HCV enzyme activities such as the HCV nucleotidyl transferase, helicase, protease or polymerase.

It is intended that combination therapies of the pharmaceutical compositions include any chemically compatible combination of a compound of this inventive group with other compounds of the inventive group or other compounds outside of the inventive group, as long as the combination does not inhibit or eliminate the anti-viral activity of the compound of this inventive group or the anti-viral activity of the pharmaceutical composition itself.

The term “interferon-alpha” as used herein means the family of highly homologous species-specific proteins that inhibit viral replication and cellular proliferation and modulate immune response. Typical suitable interferon-alphas include, but are not limited to, recombinant interferon alpha-2b, such as INTRON-A INTERFERON available from Schering Corporation, Kenilworth, N.J.; recombinant interferon alpha-2a, such as Roferon interferon available from Hofman-La Roche, Nutley, N.J.; a recombinant interferon alpha-2C, such as BEROFOR ALPHA 2 INTERFERON available from Boehringer Ingelheim Pharmaceutical, Inc., Ridgefield, Conn., interferon alpha-n1, a purified blend of natural alpha interferons, such as SUMIFERON available from Sumitomo, Japan or as Wellferon interferon alpha-n1 (INS) available from Glaxo-Wellcome Ltd., London, Great Britain, or a consensus alpha interferon, such as those described in U.S. Pat. Nos. 4,897,471 and 4,695,623 (the contents of which are hereby incorporated by reference in their entireties herein specifically examples 7, 8 or 9 thereof) and the specific product available from Amgen, Inc., Newbury Park, Calif., or interferon alpha-n3 a mixture of natural interferons made by Interferon Sciences and available from the Purdue Frederick Co., Norwalk, Conn., under the ALFERON trademark. The use of interferon alpha-2a or alpha 2b is preferred. Since interferon alpha 2b, among all interferons, has the broadest approval throughout the world for treating chronic hepatitis C infection, it is most preferred. The manufacture of interferon alpha 2b is described in U.S. Pat. No. 4,503,901 (the content of which is hereby incorporated by reference in its entirety herein).

The term “pegylated interferon” as used herein means polyethylene glycol modified conjugates of interferon, preferably interferon alpha-2a and alpha-2b. The preferred polyethylene-glycol-interferon alpha-2b conjugate is PEG.sub.12000-interferon alpha 2b. The phrase “PEG.sub.12000-IFN alpha” as used herein means conjugates such as are prepared according to the methods of International Application No. WO 95/13090 and containing urethane linkages between the interferon alpha-2a or alpha-2b amino groups and polyethylene glycol having an average molecular weight of 12000 (the content of WO 95/13090 is hereby incorporated by reference in its entirety herein).

Compounds described herein are also useful in preventing or resolving viral infections in cell, tissue or organ cultures and other in vitro applications. For example, inclusion of compounds of the invention as a supplement in cell or tissue culture growth media and cell or tissue culture components will prevent viral infections or contaminations of cultures not previously infected with viruses. Compounds described above may also be used to eliminate viruses from cultures or other biological materials infected or contaminated with viruses (e.g., blood), after a suitable treatment period, under any number of treatment conditions as determined by the skilled artisan.

Compounds of the invention can form useful salts with inorganic and organic acids such as hydrochloric, sulfuric, acetic, lactic, or the like, and with inorganic or organic bases such as sodium or potassium hydroxide, piperidine, ammonium hydroxide, or the like. The pharmaceutically acceptable salts of the compounds of formula I, II, and III, above, are prepared following procedures that are familiar to those skilled in the art.

All isomeric forms of the above-described compounds are within the scope of the invention, including, without limitation, the various isomers of the heterocyclic substituents that may be present therein, as well as tautomeric forms thereof, and cis and trans isomers. One or both of the cis and trans isomers may be synthesized about any given non-cyclic double bond and such isomers constitute preferred embodiments of the present invention.

The antiviral pharmaceutical compositions of the present invention comprise one or more of the compounds of formula I, II, and III, above, as the active ingredient, and, optionally, at least one supplemental active agent, in combination with a pharmaceutically acceptable carrier medium or auxiliary agent.

The composition may be prepared in various forms for administration, including tablets, caplets, pills or dragees, or can be filled in suitable containers, such as capsules, or, in the case of suspensions, filled into bottles. As used herein, “pharmaceutically acceptable carrier medium” includes any and all solvents, diluents, or other liquid vehicle, dispersion or suspension aids, surface active agents, isotonic agents, thickening or emulsifying agents, preservatives, solid binders, lubricants and the like, as suited to the particular dosage form desired. Remington's Pharmaceutical Sciences, Eighteenth Edition, E. W. Martin (Mack Publishing Co., Easton, Pa., 1990) discloses various carriers used in formulating pharmaceutical compositions and known techniques for the preparation thereof. Except insofar as any conventional carrier medium is incompatible with the antiviral compounds of the invention, such as by producing any undesirable biological effect or otherwise interacting in a deleterious manner with any other component(s) of the pharmaceutical composition, its use is contemplated to be within the scope of this invention.

In the pharmaceutical compositions of the invention, the active ingredient may be present in an amount of at least 0.5% and generally not more than 90% by weight, based on the total weight of the composition, including carrier medium and/or supplemental active agent(s), if any. Preferably, the proportion of active agent varies between 2-50% by weight of the composition.

Pharmaceutical organic or inorganic solid or liquid carrier media suitable for enteral or parenteral administration can be used to make up the composition. Gelatine, lactose, starch, magnesium, stearate, talc, vegetable and animal fats and oils, gum, polyalkylene glycol, or other known medicament components may all be suitable as carrier media or excipients.

The compounds of the invention may be administered using any amount and any route of administration effective for attenuating infectivity of the virus. Thus, the expression “amount effective to attenuate infectivity of virus”, as used herein, refers to a nontoxic but sufficient amount of the antiviral agent to provide the desired treatment of viral infection. The exact amount required will vary from subject to subject, depending on the species, age, and general condition of the subject, the severity of infection, the particular antiviral agent, its mode of administration, and the like.

The antiviral compounds are preferably formulated in dosage unit form for ease of administration and uniformity of dosage. “Dosage unit form” as used herein refers to a physically discrete unit of antiviral agent appropriate for the subject to be treated. Each dosage should contain the quantity of active material calculated to produce the desired therapeutic or prophylactic effect either as such, or in association with the selected pharmaceutical carrier medium and/or the supplemental active agent(s), if any. Typically, the antiviral compounds of the invention will be administered in dosage units containing from about 0.1 mg to about 500 mg of the antiviral agent by weight of the composition, with a range of about 1 mg to about 100 mg being preferred.

The compounds may be administered orally, rectally, parenterally, such as by intramuscular injection, subcutaneous injection, intravenous infusion or the like, intracisternally, intravaginally, intraperitoneally, locally, such as by powders, ointments, drops or the like, or by inhalation, such as by aerosol or the like, depending on the nature and severity of the infection being treated. Depending on the route of administration, the compounds of the invention may be administered at dosage levels of about 0.001 to about 120 mg/kg of subject body weight per day and preferably from about 0.01 to about 30 mg/kg of subject body weight per day, one or more times a day, to obtain the desired therapeutic effect. By way of example, a suitable dose for oral administration would be on the order of 20 mg/kg of body weight per day, whereas a typical dose for intravenous administration would be on the order of 10 mg/kg of body weight per day.

The compounds of the invention will typically be administered from 1 to 4 times a day so as to deliver the above-mentioned daily dosage. However, the exact regimen for administration of the compounds and compositions described herein will necessarily be dependent on the needs of the individual host being treated, the type of treatment administered and the judgment of the attending medical specialist. As used herein, the terms “host” and “subject” include both humans and animals.

In view of the inhibitory effect on viral RNA synthesis produced by the compounds of the invention, it is anticipated that these compounds will be useful not only for therapeutic treatment of virus infection, but for virus infection prophylaxis, as well. The dosages may be essentially the same, whether for treatment or prophylaxis of virus infection.

The following examples are provided to describe the invention in further detail. These examples, which set forth a preferred mode presently contemplated for carrying out the invention, are intended to illustrate and not to limit the invention.

Examples 1-3 illustrate suitable methods of synthesis of representative compounds of this invention. However, the method of synthesis is not limited to those exemplified below.

### EXAMPLE 1

### Phosphoric acid mono-(5-hydroxy-6-methyl-4-[2-methyl-1-tetrazolylimino]methyl-pyridin-3-ylmethyl)ester

A mixture of 99.0 mg (1 mmole) of 1-amino-2-methyltetrazole and 265 mg (1 mmole) of pyridoxal-5′-phosphate monohydrate in 5 ml of ethanol was heated to reflux for 30 min. A yellow solid formed. To the mixture was added 4 ml of water and the mixture cooled to room temperature and the solid collected by filtration, washed with ethanol and dried to yield 246 mg of product.

### EXAMPLE 2

### Phosphoric acid mono-(5-hydroxy-6-methyl-4-[2-napthylimino] methyl-pyridin-3-ylmethyl)ester

A mixture of 143 mg (1 mmole) of 2-aminonapthalene and 265 mg (1 mmole) of pyridoxal-5′-phosphate in 12 ml of ethanol was heated to reflux for 6 hours. The mixture was diluted with 10 ml of water and the resulting solid collected by filtration, washed with ethanol and dried.

### EXAMPLE 3

### 3-hydroxy-2-methyl-5-[(phosphonoxy)methyl]-4-pyridine carboxaldehyde phenylsulfonylhydrazone

A mixture of 172 mg (1 mmoles) of benzenesulfonylhydrazine, 124 mg (0.5 mmoles) of pyridoxal phosphate monohydrate and 19 mg (0.1 mmoles) of p-toluenesulfonic acid in 17 ml of water were heated to 70° C. for 5 hours. After cooling, water was added and the solid was collected by filtration, washed with water and dried to give 85 mg of product as a light yellow solid.

By appropriate selection of suitable reactants, other compounds of the invention may be prepared according to the above-described reaction schemes and the procedures set forth in the foregoing examples. Representative examples of further pyridine derivatives thus prepared are set forth in Table 1A, below. The compounds listed in Table 1A have the structure of formula I, in which the X moiety is —CH═N—.

TABLE 1A

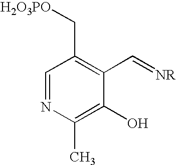

| Exam- | | |
|---|---|---|
| ple | | |
| No. | R | Compound Name |
| 4 | Ph* | Phosphoric acid mono-(5- |
| | | hydroxy-6-methyl-4- |
| | | [phenylimino]methyl- |
| | | pyridin-3-ylmethyl)ester |
| 5 | NH—S(═O)₂-2,4,6-tri CH₃—Ph | 3-Hydroxy-2-methyl-5- |
| | | [(phosphonooxy)methyl]-4- |
| | | pyridine carboxaldehyde- |
| | | 2,4,6-trimethyl phenyl |
| | | sulfonyl hydrazone |
| 6 | NH—C(═O)—Ph | 3-Hydroxy-2-methyl-5- |
| | | [(phosphonooxy)methyl]-4- |
| | | pyridine carboxaldehyde- |
| | | benzoyl hydrazone |
| 7 | NH-3-F—Ph | 3-Hydroxy-2-methyl-5- |
| | | [(phosphonooxy)methyl]-4- |
| | | pyridine carboxaldehyde- |
| | | 3-fluorophenyl hydrazone |
| 8 | NH—C(═O)—C₄H₃O | 3-Hydroxy-2-methyl-5- |
| | | [(phosphonooxy)methyl]-4- |
| | | pyridine carboxaldehyde- |
| | | 2-furoyl hydrazone |
| 9 | NH—C(═O)-4-Cl—Ph | 3-Hydroxy-2-methyl-5- |
| | | [(phosphonooxy)methyl]-4- |
| | | pyridine carboxaldehyde- |
| | | 4-chlorobenzoyl hydrazone |
| 10 | NH—C(═O)—CH₃ | 3-Hydroxy-2-methyl-5- |
| | | [(phosphonooxy)methyl]-4- |
| | | pyridine carboxaldehyde- |
| | | acetyl hydrazone |
| 11 | NH—C(═O)—H | 3-Hydroxy-2-methyl-5- |
| | | [(phosphonooxy)methyl]-4- |
| | | pyridine carboxaldehyde- |
| | | formyl hydrazone |
| 12 | NH-3-NO₂—Ph | 3-Hydroxy-2-methyl-5- |
| | | [(phosphonooxy)methyl]-4- |
| | | pyridine carboxaldehyde- |
| | | 3-nitrophenyl hydrazone |
| 13 | NH—C(═O)-3-Cl—Ph | 3-Hydroxy-2-methyl-5- |
| | | [(phosphonooxy)methyl]-4- |
| | | pyridine carboxaldehyde- |
| | | 3-chlorophenyl hydrazone |
| 14 | NH—C(═O)—C₄H₃S | 3-Hydroxy-2-methyl-5- |
| | | [(phosphonooxy)methyl]-4- |
| | | pyridine carboxaldehyde- |
| | | 2-thenoyl hydrazone |
| 15 | NH—C(═O)-4-NO₂—Ph | 3-Hydroxy-2-methyl-5- |
| | | [(phosphonooxy)methyl]-4- |
| | | pyridine carboxaldehyde- |
| | | 3-nitrobenzoyl hydrazone |
| 16 | NH—C₆H₁₁ | 3-Hydroxy-2-methyl-5- |
| | | [(phosphonooxy)methyl]-4- |
| | | pyridine carboxaldehyde- |
| | | cyclohexane hydrazone |
| 17 | NH—CH₂—Ph | 3-Hydroxy-2-methyl-5- |
| | | [(phosphonooxy)methyl]-4- |
| | | pyridine carboxaldehyde- |
| | | benzyl hydrazone |
| 18 | NH—C(═O)-2-OCH₃—Ph | 3-Hydroxy-2-methyl-5- |
| | | [(phosphonooxy)methyl]-4- |
| | | pyridine carboxaldehyde- |
| | | 2-methoxybenzoyl |
| | | hydrazone |
| 19 | NH—C(═O)-3-OC₂H₅—Ph | 3-Hydroxy-2-methyl-5- |
| | | [(phosphonooxy)methyl]-4- |
| | | pyridine carboxaldehyde- |
| | | 3-ethoxybenzoyl hydrazone |
| 20 | NH—C(═O)-2-NO₂—Ph | 3-Hydroxy-2-methyl-5- |
| | | [(phosphonooxy)methyl]-4- |
| | | pyridine carboxaldehyde- |
| | | 2-nitrobenzoyl hydrazone |
| 21 | NH-C(═O)-1-CH₃-2-pyrrole | 3-Hydroxy-2-methyl-5- |
| | | [(phosphonooxy)methyl]-4- |
| | | pyridine carboxaldehyde- |
| | | 1-methyl-2- |
| | | pyrrolecarbonyl hydrazone |
| 22 | NH—C(═O)-4-pyridine | 3-Hydroxy-2-methyl-5- |
| | | [(phosphonooxy)methyl]-4- |
| | | pyridine carboxaldehyde- |
| | | isonicotinoyl hydrazone |
| 23 | NH-2-F—Ph | 3-Hydroxy-2-methyl-5- |
| | | [(phosphonooxy)methyl]-4- |
| | | pyridine carboxaldehyde- |
| | | 2-fluorophenyl hydrazone |
| 24 | NH—CH₂—C(═O)—OC₂H₅ | 3-Hydroxy-2-methyl-5- |
| | | [(phosphonooxy)methyl]-4- |
| | | pyridine carboxaldehyde |
| | | carbethoxmethyl hydrazone |
| 25 | NH—S(═O)₂—CH₃ | 3-Hydroxy-2-methyl-5- |
| | | [(phosphonoxy)methyl]-4- |
| | | pyridine carboxaldehyde |
| | | methylsulfonylhydrazone |
| 26 | NH—S(═O)₂-4-OCH₃—Ph | 3-Hydroxy-2-methyl-5- |
| | | [(phosphonoxy)methyl-4- |
| | | pyridine carboxaldehyde- |
| | | 4-methoxyphenyl sulfonyl |
| | | hydrazone |
| 27 | NH—S(═O)₂-4-F—Ph | 3-Hydroxy-2-methyl-5- |
| | | [(phosphonoxy) methyl]-4- |
| | | pyridine carboxaldehyde- |
| | | 4-fluorophenylhydrazone |
| 28 | NH—S(═O)₂-4-CH₃—Ph | 3-Hydroxy-2-methyl-5- |
| | | [(phosphonoxy)methyl]-4- |
| | | pyridine carboxaldehyde- |
| | | 4-methylphenylhydrazone |
| 29 | NH—S(═O)₂-4- | 3-Hydroxy-2-methyl-5- |
| | CH₃—C(═O)—NH—Ph | [(phosphonoxy)methyl]-4- |
| | | pyridine carboxaldehyde- |
| | | 4-acetylaminephenyl |
| | | hydrazone |
| 30 | NH—S(═O)₂-4-t-butyl—Ph | 3-Hydroxy-2-methyl-5- |
| | | [(phosphonoxy)methyl]-4- |
| | | pyridine carboxaldehyde- |
| | | 4-t-butylphenylhydrazone |
| 31 | NH—S(═O)₂-2-N(CH₃)₂-6- | 3-Hydroxy-2-methyl-5- |
| | Nap** | [(phosphonoxy)methyl]-4- |
| | | pyridine carboxaldehyde- |
| | | 2-dimethylamino napthyl |
| | | hydrazone |

*Ph = phenyl (C₆H₅—)
**Nap = napthyl (C₁₀H₇—)

### EXAMPLE 32

### Phosphoric acid mono-(5-hydroxy-6-methyl-4-styryl-pyridin-3-ylmethyl)ester

106 mg (2.6 mmol) of sodium hydride (as a 60% dispersion in mineral oil) was added to 10 ml anhydrous DMSO and allowed to stir at room temperature, under argon, for one hour. To the mixture was added 807 mg (2.1 mmol) of benzyltriphenylphosphonium chloride in three portions over 15 minutes. A second solution was prepared by dissolving 500 mg (1.9 mmol) of pyridoxal-5-phosphate monohydrate in 5 ml DMSO with heating. 3 Å molecular sieves were added to this solution to exclude water. This second solution was added to the reaction mixture over 10 minutes, and the resulting mixture was stirred at room temperature overnight.

The mixture was poured over 100 ml of water, and 1 M NaOH was added until a pH of 9-10 was reached. The mixture was extracted first with t-butyl methyl ether and then with methylene chloride. The aqueous layer was acidified with acetic acid and extracted with ethyl acetate. This aqueous layer was saturated with sodium chloride. The resulting precipitate was collected by filtration, washed with a small amount of water and t-butyl methyl ether, and dried to yield 140 mg of product.

Additional representative examples of pyridine derivatives of the invention are set forth in Table 1B, below.

The compounds listed in Table 1B have the structure of formula I, in which the X moiety is —CH═CRₐ—.

TABLE 1B

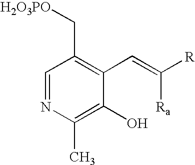

| Example No. | Rₐ | R | Compound Name |
|---|---|---|---|
| 33 | H | H | Phosphoric acid mono-(5-hydroxy-6- |
| | | | methyl-4-vinyl-pyridin-3- |
| | | | ylmethyl) ester |
| 34 | H | CH₃ | Phosphoric acid mono-(5-hydroxy-6- |
| | | | methyl-4-propenyl-pyridin-3-ylmethyl) |
| | | | ester |
| 35 | CH₃ | CH₃ | Phosphoric acid mono-(5-hydroxy-6- |
| | | | methyl-4-(2-methyl-propenyl)-pyridin- |
| | | | 3-ylmethyl)ester |
| 36 | CH₃ | Ph | Phosphoric acid mono-(5-hydroxy-6- |
| | | | methyl-4-(2-phenyl-propenyl)-pyridin- |
| | | | 3-ylmethyl)ester |
| 37 | H | PhMe | Phosphoric acid mono-(5-hydroxy-6- |
| | | | methyl-4-(4-methylstryl)-pyridin-3- |
| | | | ylmethyl)ester |
| 38 | H | Ph CF₃ | Phosphoric acid mono-(5-hydroxy-6- |
| | | | methyl-4-(4-trifluoromethylstryl)- |
| | | | pyridin-3-ylmethyl)ester |
| 39 | H | Ph—OCF₃ | Phosphoric acid mono-(5-hydroxy-6- |
| | | | methyl-4-(4-trifluoromethoxystryl)- |
| | | | pyridin-3-ylmethyl)ester |
| 40 | H | PhF | Phosphoric acid mono-(5-hydroxy-6- |
| | | | methyl-4-(4-fluorostryl)-pyridin-3- |
| | | | ylmethyl)ester |

### EXAMPLE 41

### Inhibition of Viral RNA Replication

The discovery of inhibitors of viral polymerases and related proteins generally requires the evaluation of large numbers of chemical compounds or mixtures of chemical compounds. Thus, an assay for the polymerase activity that is capable of high volume screening, in other words, a high-throughput assay, is desirable. There are a variety of assay methodologies well known to the trained artisan that allow the efficient screening of large numbers of samples. See, for example, Cole, J L, Meth Enzymology, 275: 310-328 (1996). Any one of these assays may be suitable in the case of a viral RdRp activity.

One approach for measuring viral RdRp activity in the case of viruses of the Flaviviridae utilizes a purified recombinant NS5 protein in an in vitro RdRp assay. For example, Behrens et al. [EMBO J., 15: 12-22 (1996)] and Lohmann et al. [J Virol, 71:8416-8428 (1997)] describe the baculovirus expression, purification and enzymatic activity of the HCV NS5B RdRp. The bacterial expression, purification and enzymatic activity of the HCV NS5B RdRp protein has been disclosed in PCT/US96/15571 [WO 97/12033] and by Yuan et al. [Bioochem Biophys Res Comm, 232:231-235 (1997)]. In a further example, Collett, PCT/US99/07404, [WO 99/51781], which is commonly owned with the present application, discloses compositions comprising functional HCV NS5B sequences and their use in identifying compounds useful in the treatment of hepacivirus infections. As with the above examples for the HCV RdRp, bacterially-expressed dengue flavivirus NS5 protein has been purified and shown to exhibit RdRp activity [Tan et al., Virology, 216: 317-325 (1996)], as has the NS5B protein of the pestivirus BVDV purified from recombinant baculovirus-infected cells [zhong et al., J. Virol., 72: 9365-9369 (1998)].

By way of example, the inhibitory activity of candidate compounds within the scope of this invention was demonstrated in in vitro RdRp assays using NS5 proteins prepared essentially according to Collett, PCT/US99/07404, the entire disclosure of which is incorporated by reference herein. Purified NS5 proteins are incubated in standard RdRp reaction mixtures. Such reaction mixtures generally consist of buffers, salts, cations, reducing agents and the like, as well as nucleoside triphosphates and an RNA template-primer. Variations in the individual components of such reaction mixtures may be required to accommodate the particular reaction preferences of individual NS5 proteins. Such variations are well known to the trained artisan.

Representative compounds within the scope of the present invention, as shown in Examples 1-3 and the foregoing table, were evaluated for antiviral activity in this assay. Inhibitory activity of the compounds tested was expressed in IC₅₀ values. IC₅₀ values represent the concentration of the compound at which 50% of the RdRp activity is inhibited. The results of the assay for inhibition of RdRp activity in at least one virus of the Flaviviridae family for the compounds tested revealed IC₅₀ values ranging from 0.08 to about 30 μM. The low concentrations of test compounds capable of achieving 50% inhibition of the RdRp activity indicate that the compounds of the invention are effective at inhibiting RNA synthesis by viral RdRp enzymes involved in Flaviviridae replication.

The following table contains examples of prodrugs of Formula III which may be synthesized using conventional chemistry knowledge. See, e.g., the References section, below. In the following examples, X, R, and R₁ are as previously defined.

TABLE 2

| | |
|---|---|
| | (II) |
| 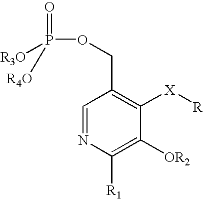 | |

| | | | |
|---|---|---|---|
| Ex- | | | |
| am- | | | |
| ple | | | |
| No. | R₂ | R₃ | R₄ |

| | | | |
|---|---|---|---|
| 42 | —C(═O)CH₃ | H | H |
| 43 | —C(═O)Ph | H | H |
| 44 | —C(═O)CH₃ | A substituent | H |
| | | selected from the | |
| | | group consisting of | |
| | | C₁-C₆-alkyl, | |
| | | cyclohexyl, phenyl, | |
| | | (4-methyl )phenyl, | |
| | | (4-n-propyl)phenyl, | |
| | | (4- | |
| | | isopropyl)phenyl, | |
| | | (3,4- | |
| | | dimethyl)phenyl, | |
| | | (3,5,- | |
| | | dimethyl)phenyl, | |
| | | indanyl, (3- | |
| | | methoxyphenyl) and | |
| | | (4-methoxy)phenyl. | |
| 45 | —C(═O)Ph | A substituent | H |
| | | selected from the | |
| | | group consisting of | |
| | | C₁-C₆-alkyl, | |
| | | cyclohexyl, phenyl, | |
| | | (4-methyl)phenyl, | |
| | | (4-n-propyl)phenyl, | |
| | | (4- | |
| | | isopropyl)phenyl, | |
| | | (3,4- | |
| | | dimethyl)phenyl, | |
| | | (3,5,- | |
| | | dimethyl)phenyl, | |
| | | indanyl, (3- | |
| | | methoxyphenyl) and | |
| | | (4-methoxy)phenyl. | |
| 46 | —C(═O)CH₃ | phenyl | H |
| 47 | —C(═O)Ph | phenyl | H |
| 48 | —C(═O)CH₃ | ethyl | H |
| 49 | —C(═O)Ph | ethyl | H |
| 50 | —C(═O)CH₃ | cyclohexyl | H |
| 51 | —C(═O)Ph | cyclohexyl | H |
| 52 | —C(═O)CH₃ | phenyl | phenyl |
| 53 | —C(═O)Ph | phenyl | phenyl |
| 54 | —C(═O)CH₃ | A substituent | A substituent |
| | | selected from the | selected from the |
| | | group consisting of | group consisting |
| | | C₁-C₆-alkyl, | of C₁-C₆-alkyl, |
| | | cyclohexyl, phenyl, | cyclohexyl, |
| | | (4-methyl)phenyl, | phenyl, (4- |
| | | (4-n-propyl)phenyl, | methyl)phenyl, |
| | | (4- | (4-n- |
| | | isopropyl)phenyl, | propyl)phenyl, |
| | | (3,4- | (4- |
| | | dimethyl)phenyl, | isopropyl)phenyl, |
| | | (3,5,- | (3,4- |
| | | dimethyl)phenyl, | dimethyl)phenyl, |
| | | indanyl, (3- | (3,5,- |
| | | methoxyphenyl) and | dimethyl)phenyl, |
| | | (4-methoxy)phenyl. | indanyl, (3- |
| | | | methoxyphenyl) |
| | | | and (4- |
| | | | methoxy)phenyl. |
| 55 | —C(═O)Ph | A substituent | A substituent |
| | | selected from the | selected from the |
| | | group consisting of | group consisting |
| | | C₁-C₆-alkyl, | of C₁-C₆-alkyl, |
| | | cyclohexyl, phenyl, | cyclohexyl, |
| | | (4-methyl)phenyl, | phenyl, (4- |
| | | (4-n-propyl)phenyl, | methyl)phenyl, |
| | | (4- | (4-n- |
| | | isopropyl) phenyl, | propyl)phenyl, |
| | | (3,4- | (4- |
| | | dimethyl)phenyl, | isopropyl)phenyl, |
| | | (3,5,- | (3,4- |
| | | dimethyl)phenyl, | dimethyl)phenyl, |
| | | indanyl, (3- | (3,5,- |
| | | methoxyphenyl) and | dimethyl)phenyl, |
| | | (4-methoxy)phenyl. | indanyl, (3- |
| | | | methoxyphenyl) |
| | | | and (4- |
| | | | methoxy)phenyl. |
| 56 | Residue of | H | H |
| | an amino | | |
| | acid | | |
| | selected | | |
| | from the | | |
| | group | | |
| | consisting | | |
| | of | | |
| | alanine, | | |
| | valine | | |
| | leucine, | | |
| | isoleucine, | | |
| | proline, | | |
| | phenylalanine, | | |
| | tryptophan | | |
| | and | | |
| | methionine | | |
| 57 | Alanine | H | H |
| 58 | Valine | H | H |
| 59 | Leucine | H | H |
| 60 | Residue of | H | H |
| | an amino | | |
| | acid | | |
| | selected | | |
| | from the | | |
| | group | | |
| | consisting | | |
| | of | | |
| | glycine, | | |
| | serine, | | |
| | threonine, | | |
| | cysteine, | | |
| | tyrosine, | | |
| | asparagines | | |
| | and | | |
| | glutamine. | | |
| 61 | Glycine | H | H |
| 62 | Serine | H | H |
| 63 | Threonine | H | H |
| 64 | Residue of | H | H |
| | an amino | | |
| | acid | | |
| | selected | | |
| | from the | | |
| | group | | |
| | consisting | | |
| | of | | |
| | aspartic | | |
| | acid, | | |
| | glutamic | | |
| | acid, | | |
| | lysine, | | |
| | arginine | | |
| | and | | |
| | histidine. | | |
| 65 | Aspartic | H | H |
| | acid | | |
| 66 | Glutamic | H | H |
| | acid | | |
| 67 | Lysine | H | H |
| 68 | Residue of | A substituent | H |
| | an amino | selected from the | |
| | acid | group consisting of | |
| | selected | C₁-C₆-alkyl, | |
| | from the | cyclohexyl, phenyl, | |
| | group | (4-methyl)phenyl, | |
| | consisting | (4-n-propyl)phenyl, | |
| | of | (4- | |
| | alanine, | isopropyl)phenyl, | |
| | valine | (3,4- | |
| | leucine, | dimethyl)phenyl, | |
| | isoleucine | (3,5,- | |
| | proline, | dimethyl)phenyl, | |
| | phenyl- | indanyl, (3- | |
| | alanine, | methoxyphenyl) and | |
| | tryptophan | (4-methoxy)phenyl. | |
| | and | | |
| | methionine | | |
| 69 | Alanine | phenyl | H |
| 70 | Valine | phenyl | H |
| 71 | Leucine | phenyl | H |
| 72 | Residue of | A substituent | H |
| | an amino | selected from the | |
| | acid | group consisting of | |
| | selected | C₁-C₆-alkyl, | |
| | from the | cyclohexyl, phenyl, | |
| | group | (4-methyl )phenyl, | |
| | consisting | (4-n-propyl)phenyl, | |
| | of | (4- | |
| | glycine, | isopropyl)phenyl, | |
| | serine, | (3,4- | |
| | threonine, | dimethyl)phenyl, | |
| | cysteine, | (3,5,- | |
| | tyrosine, | dimethyl)phenyl, | |
| | asparagines | indanyl, (3- | |
| | and | methoxyphenyl) and | |
| | glutamine. | (4-methoxy)phenyl. | |
| 73 | Glycine | phenyl | H |
| 74 | Serine | phenyl | H |
| 75 | Threonine | phenyl | H |
| 76 | Residue of | A substituent | H |
| | an amino | selected from the | |
| | acid | group consisting of | |
| | selected | C₁-C₆-alkyl, | |
| | from the | cyclohexyl, phenyl, | |
| | group | (4-methyl)phenyl, | |
| | consisting | (4-n-propyl)phenyl, | |
| | of | (4- | |
| | aspartic | isopropyl)phenyl, | |
| | acid, | (3,4- | |
| | glutamic | dimethyl)phenyl, | |
| | acid, | (3,5,- | |
| | lysine, | dimethyl)phenyl, | |
| | arginine | indanyl, (3- | |
| | and | methoxyphenyl) and | |
| | histidine. | (4-methoxy)phenyl. | |
| 77 | Aspartic | phenyl | H |
| | acid | | |
| 78 | Glutamac | phenyl | H |
| | acid. | | |
| 79 | Lysine | phenyl | H |
| 80 | Residue of | A substituent | A substituent |
| | an amino | selected from the | selected from the |
| | acid | group consisting of | group consisting |
| | selected | C₁-C₅-alkyl, | of C₁-C₆-alkyl, |
| | from the | cyclohexyl, phenyl, | cyclohexyl, |
| | group | (4-methyl)phenyl, | phenyl, (4- |
| | consisting | (4-n-propyl)phenyl, | methyl)phenyl, |
| | of | (4- | (4-n- |
| | alanine, | isopropyl)phenyl, | propyl)phenyl, |
| | valine | (3,4- | (4- |
| | leucine, | dimethyl)phenyl, | isopropyl)phenyl, |
| | isoleucine | (3,5,- | (3,4- |
| | proline, | dimethyl)phenyl, | dimethyl)phenyl, |
| | phenyl- | indanyl, (3- | (3,5,- |
| | alanine, | methoxyphenyl) and | dimethyl)phenyl, |
| | tryptophan | (4-methoxy)phenyl. | indanyl, (3- |
| | and | | methoxyphenyl) |
| | methionine. | | and (4- |
| | | | methoxy)phenyl. |
| 81 | Alanine | phenyl | phenyl |
| 82 | Valine | phenyl | phenyl |
| 83 | Leucine | phenyl | phenyl |
| 84 | Residue of | A substituent | A substituent |
| | an amino | selected from the | selected from the |
| | acid | group consisting of | group consisting |
| | selected | C₁-C₆-alkyl, | of C₁-C₆-alkyl, |
| | from the | cyclohexyl, phenyl, | cyclohexyl, |
| | group | (4-methyl)phenyl, | phenyl, (4- |
| | consisting | (4-n-propyl)phenyl, | methyl) phenyl, |
| | of | (4- | (4-n- |
| | glycine, | isopropyl)phenyl, | propyl)phenyl, |
| | serine, | (3,4- | (4- |
| | threonine, | dimethyl)phenyl, | isopropyl)phenyl, |
| | cysteine, | (3,5,- | (3,4- |
| | tyrosine, | dimethyl)phenyl, | dimethyl)phenyl, |
| | asparagines | indanyl, (3- | (3,5,- |
| | and | methoxyphenyl) and | dimethyl)phenyl, |
| | glutamine. | (4-methoxy)phenyl. | indanyl, (3- |
| | | | methoxyphenyl) |
| | | | and (4- |
| | | | methoxy)phenyl. |
| 85 | Glycine | phenyl | phenyl |
| 86 | Serine | phenyl | phenyl |
| 87 | Threonine | phenyl | phenyl |
| 88 | Residue of | A substitutent | A substituent |
| | an amino | selected from the | selected from the |
| | acid | group consisting of | group consisting |
| | selected | C₁-C₆-alkyl, | of C₁-C₆-alkyl, |
| | from the | cyclohexyl, phenyl, | cyclohexyl, |
| | group | (4-methyl)phenyl, | phenyl, (4- |
| | consisting | (4-n-propyl)phenyl, | methyl)phenyl, |
| | of | (4- | (4-n- |
| | aspartic | isopropyl)phenyl, | propyl)phenyl, |
| | acid, | (3,4- | (4- |
| | glutamic | dimethyl)phenyl, | isopropyl)phenyl, |
| | acid, | (3,5,- | (3,4- |
| | lysine, | dimethyl)phenyl, | dimethyl)phenyl, |
| | arginine | indanyl, (3- | (3,5,- |
| | and | methoxyphenyl) and | dimethyl)phenyl, |
| | histidine. | (4-methoxy)phenyl. | indanyl, (3- |
| | | | methoxyphenyl) |
| | | | and (4- |
| | | | methoxy) phenyl. |
| 89 | Aspartic | phenyl | phenyl |
| | acid | | |
| 90 | Glutanlic | pheny | phenyl |
| | acid | | |
| 91 | Lysine | phenyl | phenyl |
| 92 | Residue of | A substituent | H |
| | an amino | selected from the | |
| | acid | group consisting of | |
| | selected | CH₂O(CO)t-butyl, | |
| | from the | CH₂O (CO)isopropyl, | |
| | group | CH(Me)O(CO)ethyl, | |
| | consisting | CH(iPr)O(CO)ethyl, | |
| | of | CH(cHex)O(CO)ethyl, | |
| | alanine, | CH(iPr)O(CO)iso- | |
| | valine | propyl, and | |
| | leucine, | CH(iPr)O(CO)n- | |
| | isoleucine, | heptyl. | |
| | proline, | | |
| | phenyl- | | |
| | alanine, | | |
| | tryptophan | | |
| | and | | |
| | methionine. | | |
| 93 | Alanine | CH(iPr)O(CO)n- | H |
| | | heptyl | |
| 94 | Valine | CH(iPr)O(CO)n- | H |
| | | heptyl | |
| 95 | Leucine | CH(iPr)O(CO)n- | H |
| | | heptyl | |
| 96 | Residue of | A substituent | H |
| | an amino | selected from the | |
| | acid | group consisting of | |
| | selected | CH₂O(CO)t-butyl, | |
| | from the | CH₂O(CO)isopropyl, | |
| | group | CH(Me)O(CO)ethyl, | |
| | consisting | CH(iPr)O(CO)ethyl, | |
| | of | CH(cHex)O(CO)ethyl, | |
| | glycine, | CH(iPr)O(CO)iso- | |
| | serine, | propyl, and | |
| | threonine, | CH(iPr)O(CO)n- | |
| | cysteine, | heptyl | |
| | tyrosine, | | |
| | asparagines | | |
| | and | | |
| | glutamine. | | |
| 97 | Glycine | CH(iPr)O(CO)n- | H |
| | | heptyl | |
| 98 | Serine | CH(iPr)O(CO)n- | H |
| | | heptyl | |
| 99 | Threonine | CH(iPr)O(CO)n- | H |
| | | heptyl | |
| 100 | Residue of | A substituent | H |
| | an amino | selected from the | |
| | acid | group consisting of | |
| | selected | CH₂O(CO)t-butyl, | |
| | from the | CH₂O(CO)isopropyl, | |
| | group | CH(Me)O(CO)ethyl, | |
| | consisting | CH(iPr)O(CO)ethyl, | |
| | of | CH(cHex)O(CO)ethyl, | |
| | aspartic | CH(iPr)O(CO)iso- | |
| | acid, | propyl, and | |
| | glutamic | CH(iPr)O(CO)n- | |
| | acid, | heptyl | |
| | lysine, | | |
| | arginine | | |
| | and | | |
| | histidine. | | |
| 101 | Aspartic | CH(iPr)O(CO)n- | H |
| | acid | heptyl | |
| 102 | Glutamic | CH(ipr)O(CO)n- | H |
| | acid | heptyl | |
| 103 | Lysine | CH(iPr)O(CO)n- | H |
| | | heptyl | |
| 104 | Residue of | A substituent | A substituent |
| | an amino | selected from the | selected from the |
| | acid | group consisting of | group consisting |
| | selected | CH₂O(CO)t-butyl, | of CH₂O(CO)t- |
| | from the | CH₂O(CO)isopropyl, | butyl, |
| | group | CH(Me)O(CO)ethyl, | CH₂O(CO)iso- |
| | consisting | CH(ipr)O(CO)ethyl, | propyl, |
| | of | CH(cHex)O(CO)ethyl, | CH(Me)O(CO)ethyl, |
| | alanine, | CH(iPr)O(CO)iso- | CH(iPr)O(CO)ethyl, |
| | valine | propyl, and | CH(cHex)O(CO)ethyl, |
| | leucine, | CH(iPr)O(CO)n- | CH(iPr)O(CO)iso- |
| | isoleucine | heptyl | propyl, and |
| | praline, | CH(ipr)O(CO)n- | |
| | phenyl | heptyl | |
| | alanine | | |
| | tryptophan | | |
| | and | | |
| | methionine. | | |
| 105 | Alanine | CH(iPr)O(CO)n- | CH(iPr)O(CO)n- |
| | | heptyl | heptyl |
| 106 | Valine | CH(iPr)O(CO)n- | CH(iPr)O(CO)n- |
| | | heptyl | heptyl |
| 107 | Leucine | CH(ipr)O(CO)n- | CH(iPr)O(CO)n- |
| | | heptyl | heptyl |
| 108 | Residue of | A substituent | A substituent |
| | an amino | selected from the | selected from the |
| | acid | group consisting of | group consisting |
| | selected | CH₂O(CO)t-butyl, | of CH₂O(CO)t- |
| | from the | CH₂O(CO)isopropyl, | butyl, |
| | group | CH(Me)O(CO)ethyl, | CH₂O(CO)iso- |
| | consisting | CH(ipr)O(CO)ethyl, | propyl, |
| | of | CH(cHex)O(CO)ethyl, | CH(Me)O(CO)ethyl, |
| | glycine, | CH(iPr)O(CO)iso- | CH(ipr)O(CO)ethyl, |
| | serine, | propyl, and | CH(cHex)O(CO)ethyl, |
| | threonine, | CH(iPr)O(CO)n- | CH(iPr)O(CO)iso- |
| | cysteine, | heptyl | propyl, and |
| | tyrosine, | CH(iPr)O(CO)n- | |
| | asparagines | heptyl | |
| | and | | |
| | glutainine. | | |
| 109 | Glycine | CH(iPr)O(CO)n- | CH(ipr)O(CO)n- |
| | | heptyl | heptyl |
| 110 | Serine | CH(iPr)O(CO)n- | CH(iPr)O(CO)n- |
| | | heptyl | heptyl |
| 111 | Threonine | CH(iPr)O(CO)n- | CH(iPr)O(CO)n- |
| | | heptyl | heptyl |
| 112 | Residue of | A substituent | A substituent |
| | an amino | selected from the | selected from the |
| | acid | group consisting of | group consisting |
| | selected | CH₂O(CO)t-butyl, | of CH₂O(CO)t- |
| | from the | CH₂O(CO)isopropyl, | butyl, |
| | group | CH(Me)O(CO)ethyl, | CH₂O(CO)iso- |
| | consisting | CH(iPr)O(CO)ethyl, | propyl, |
| | of | CH(cHex)O(CO)ethyl, | CH(Me)O(CO)ethyl, |
| | aspartic | CH(iPr)O(CO)iso- | CH(ipr)O(CO)ethyl, |
| | acid, | propyl, and | CH(cHex)0(CO)ethyl, |
| | glutamic | CH(iPr)O(CO)n- | CH(iPr)O(CO) iso- |
| | acid, | heptyl | propyl, and |
| | lysine, | | CH(iPr)O(CO)n- |
| | arginine | | heptyl |
| | and | | |
| | histidine. | | |
| 113 | Aspartic | CH(iPr)O(CO)n- | CH(iPr)O(CO)n- |
| | acid | heptyl | heptyl |
| 114 | Glutamic | CH(iPr)O(CO)n- | CH(ipr)O(CO)n- |
| | acid | heptyl | heptyl |
| 115 | Lysine | CH(iPr)O(CO)n- | CH(ipr)O(CO)n- |
| | | heptyl | heptyl |
| 116 | H | A substituent | H |
| | | selected from the | |
| | | group consisting of | |
| | | C₁-C₆-alkyl, | |
| | | cyclohexyl, phenyl, | |
| | | (4-methyl)phenyl, | |
| | | (4-n-propyl)phenyl, | |
| | | (4- | |
| | | isopropyl)phenyl, | |
| | | (3,4- | |
| | | dimethyl)phenyl, | |
| | | (3,5,- | |
| | | dimethyl)phenyl, | |
| | | indanyl,(3- | |
| | | methoxyphenyl) and | |
| | | (4-methoxy)phenyl. | |
| 117 | H | A substituent | A substituent |
| | | selected from the | selected from the |
| | | group consisting of | group consisting |
| | | C₁-C₆-alkyl, | of C₁-C₆-alkyl, |
| | | cyclohexyl, phenyl, | cyclohexyl, |
| | | (4-methyl)phenyl, | phenyl, (4- |
| | | (4-n-propyl)phenyl, | methyl)phenyl, |
| | | (4- | (4-n- |
| | | isopropyl)phenyl, | propyl)phenyl, |
| | | (3,4- | (4- |
| | | dimethyl)phenyl, | isopropyl)phenyl, |
| | | (3,5,- | (3,4- |
| | | dimethyl)phenyl, | dimethyl)phenyl, |
| | | indanyl, (3- | (3,5,- |
| | | methoxyphenyl) and | dimethyl)phenyl, |
| | | (4-methoxy)phenyl. | indanyl, (3- |
| | | | methoxyphenyl) |
| | | | and (4- |
| | | | methoxy)phenyl. |
| 118 | H | phenyl | H |
| 119 | H | phenyl | ethyl |
| 120 | H | ethyl | H |
| 121 | H | ethyl | phenyl |
| 122 | H | cyclohexyl | H |
| 123 | H | cyclohexyl | ethyl |
| 124 | H | phenyl | phenyl |
| 125 | H | phenyl | cyclohexyl |
| 126 | H | A substituent | cyclohexyl |
| | | selected from the | |
| | | group consisting of | |
| | | C₁-C₆-alkyl, | |
| | | cyclohexyl, phenyl, | |
| | | (4-methyl)phenyl, | |
| | | (4-n-propyl)phenyl, | |
| | | (4- | |
| | | isopropyl)phenyl, | |
| | | (3,4- | |
| | | dimethyl)phenyl, | |
| | | (3,5,- | |
| | | dimethyl)phenyl, | |
| | | indanyl, (3- | |
| | | methoxyphenyl) and | |
| | | (4-methoxy) phenyl. | |
| 127 | H | A substituent | (4-methyl)phenyl |
| | | selected from the | |
| | | group consisting of | |
| | | C₁-C₆-alkyl, | |
| | | cyclohexyl, phenyl, | |
| | | (4-methyl)phenyl, | |
| | | (4-n-propyl)phenyl, | |
| | | (4- | |
| | | isopropyl)phenyl, | |
| | | (3,4- | |
| | | dimethyl)phenyl, | |
| | | (3,5,- | |
| | | dimethyl)phenyl, | |
| | | indanyl, (3- | |
| | | methoxyphenyl) and | |
| | | (4-methoxy)phenyl. | |
| 128 | H | A substituent | ethyl |
| | | selected from the | |
| | | group consisting of | |
| | | C₁-C₆-alkyl, | |
| | | cyclohexyl, phenyl, | |
| | | (4-methyl)phenyl, | |
| | | (4-n-propyl)phenyl, | |
| | | (4- | |
| | | isopropyl)phenyl, | |
| | | (3,4- | |
| | | dimethyl)phenyl, | |
| | | (3,5,- | |
| | | dimethyl)phenyl, | |
| | | indanyl, (3- | |
| | | methoxyphenyl) and | |
| | | (4-methoxy)phenyl. | |
| 129 | H | ethyl | ethyl |
| 130 | H | cyclohexyl | cyclohexyl |
| 131 | H | (4-methyl)phenyl | (4-methyl)phenyl |
| 132 | H | A substituent | phenyl |
| | | selected from the | |
| | | group consisting of | |
| | | C₁-C₆-alkyl, | |
| | | cyclohexyl, phenyl, | |
| | | (4-methyl)phenyl, | |
| | | (4-n-propyl)phenyl, | |
| | | (4- | |
| | | isopropyl)phenyl, | |
| | | (3,4- | |
| | | dimethyl)phenyl, | |
| | | (3,5,- | |
| | | dimethyl)phenyl, | |
| | | indanyl, (3- | |
| | | methoxyphenyl) and | |
| | | (4-methoxy)phenyl. | |
| 133 | H | (4-n-propyl)phenyl | (4-n- |
| | | | propyl)phenyl |
| 134 | H | (4-isopropyl)phenyl | (4- |
| | | | isopropyl)phenyl |
| 135 | H | (3,4- | (3,4- |
| | | dimethyl)phenyl | dimethyl)phenyl |
| 136 | H | A substituent | C₁-C₆-alkyl |
| | | selected from the | |
| | | group consisting of | |
| | | C₁-C₆-alkyl, | |
| | | cyclohexyl, phenyl, | |
| | | (4-methyl)phenyl, | |
| | | (4-n-propyl)phenyl, | |
| | | (4- | |
| | | isopropyl)phenyl, | |
| | | (3,4- | |
| | | dimethyl)phenyl, | |
| | | (3,5,- | |
| | | dimethyl)phenyl, | |
| | | indanyl, (3- | |
| | | methoxyphenyl) and | |
| | | (4-methoxy)phenyl. | |
| 137 | H | (3,5-dimethyl) | (3,5-dimethyl) |
| | | phenyl indanyl | phenyl indanyl |
| 138 | H | C₁-C₆-alkyl | C₁-C₆-alkyl |
| 139 | H | (4-methoxy)phenyl | (4-methoxy)phenyl |
| 140 | H | A substituent | H |
| | | selected from the | |
| | | group consisting of | |
| | | CH₂O(CO)t-butyl, | |
| | | CH₂O(CO)isopropyl, | |
| | | CH(Me)O(CO)ethyl, | |
| | | CH(iPr)O(CO)ethyl, | |
| | | CH(cHex)O(CO)ethyl, | |
| | | CH(iPr)O(CO)- | |
| | | isopropyl, and | |
| | | CH(iPr)O(CO)n- | |
| | | heptyl | |
| 141 | H | CH(iPr)O(CO)n- | H |
| | | heptyl | |
| 142 | H | CH(ipr)O(CO)n- | H |
| | | heptyl | |
| 143 | H | CH(iPr)O(CO)n- | H |
| | | heptyl | |

Although the present invention has been described and exemplified in terms of certain preferred embodiments, other embodiments will be apparent to those skilled in the art. The invention is, therefore, not limited to the particular embodiments described and exemplified, but is capable of modification or variation without departing from the spirit of the invention, the full scope of which is delineated by the appended claims.

### REFERENCES

The following publications are incorporated by reference herein in their entirety.
1. Lombaert et al., J. Med. Chem., Vol. 37, p. 498-511 (1994).
2. Vepsalainen, Tet. Letters, Vol. 40, p. 8491-8493 (1999).
3. Sawaki et al., The Effect of Pyridoxal Phosphate on Type C Hepatitis, Medicine and Biology, Vol. 135, No. 1, p. 13-15 (1997).
4. Sawaki et al., Effect of Pyridoxal Phosphate Administration on Hepatitis C Virus RNA in Patients with Hepatitis Type C, Vol. 135, No. 4, p. 149-151 (1997).

1. A compound having the formula: 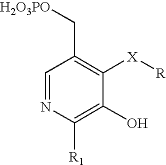
wherein X represents a divalent linking moiety selected from the group —CH═N— or —CH═CRₐ—;
R is a radical selected from the group consisting of an unsubstituted or substituted alkyl (C₁-C₆) radical, an unsubstituted or substituted aryl (C₆-C₁₄) radical, an unsubstituted or substituted aralkyl (C₇-C₁₅) radical, an unsubstituted or substituted heterocyclic radical, or a radical of the formula —NRₐ—X′—R_{b}, wherein X′ represents a valence bond or a divalent linking moiety selected from the group of —C(═O)—, —S(═O)₂— or —(CH₂)ₙ—, n being an integer from 1 to 6;
Rₐ represents hydrogen or an unsubstituted or substituted alkyl (C₁-C₆) radical;
R_{b} represents hydrogen, an unsubstituted or substituted alkyl (C₁-C₆) radical, an unsubstituted or substituted aryl (C₆-C₁₄) radical, an unsubstituted or substituted aralkyl (C₇-C₁₆) radical, an unsubstituted or substituted heterocyclic radical, an unsubstituted or substituted alicyclic (C₅-C₇) radical or a carbalkoxy radical;
R₁ represents an unsubstituted or substituted alkyl (C₁-C₆) radical;
said heterocyclic radical represented by R or R_{b} being at least one selected from the group consisting of furan, thiophene, pyrrole, tetrazole, pyridine, piperidine, morpholine, pyrazole, pyridazine, triazole, pyrimidine, oxadiazole, thiadiazole, oxazole, isoxazole, isothiazole, and azepane; said alkyl radical substituent(s) being at least one selected from the group consisting of carboxy, hydroxy, alkoxy, amino, alkylamino, dialkylamino, thiol and alkylthio; said aryl radical substituent(s) and said aralkyl radical substituent(s) being at least one selected from the group consisting of a straight or branched chain, saturated or unsaturated aliphatic group having 1-6 carbon atoms, halogen, nitro, carboxy, hydroxy, hydroxyalkyl, perhaloalkyl, monohaloalkyl, dihaloalkyl, alkoxy, perhaloalkoxy, phenylalkoxy, acyl, acyloxy, acyloxyalkyl, cyano, carbalkoxy, thiol, alkylthio, alkylsulfinyl, alkylsulfonyl, amino, alkylamino, dialkylamino, aminoalkyl, alkylaminoalkyl, dialkylaminoalkyl, sulfonamido, carboxamido, and alkanoylamino; said heterocyclic radical substituent(s) and said alicyclic radical substituents(s) being at least one selected from the group consisting of a straight or branched chain, saturated or unsaturated aliphatic group having 1-6 carbon atoms, halogen, perhaloalkyl, monohaloalkyl, dihaloalkyl, alkoxy, acyl, acyloxy, acyloxyalkyl, phenylalkoxy, hydroxy, hydroxyalkyl, alkylsulfonate, thiol, alkylthio, alkylsulfinyl, alkylsulfonyl, nitro, carboxy, carbalkoxy, or an unsubstituted and substituted aryl (C₆-C₁₄) radical; the isomeric forms of said compound and the pharmaceutically acceptable salts of said compound.

2. A pharmaceutical composition for treating or preventing viral infections, said composition comprising a compound as claimed in claim 1 in an amount effective to attenuate viral infectivity, and a pharmaceutically acceptable carrier medium.

3. A pharmaceutical composition as claimed in claim 2 further comprising at least one supplemental active agent selected from the group of interferon, a pegylated interferon, ribavirin, protease inhibitors, polymerase inhibitors, small interfering RNA compounds, anti-sense compounds, nucleotide analogs, nucleoside analogs, immunoglobulins, immunomodulators, hepatoprotectants, anti-inflammatory agents, antibiotics, antivirals, and anti-infective compounds.

4. A compound for treating or preventing viral infection and disease associated with said infection in living hosts, said compound having the formula: 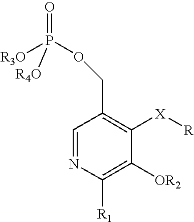
wherein X represents a divalent linking moiety selected from the group —CH═N— or —CH═CRₐ—;
R is a radical selected from the group consisting of an unsubstituted or substituted alkyl (C₁-C₆) radical, an unsubstituted or substituted aryl (C₆-C₁₄) radical, an unsubstituted or substituted aralkyl (C₇-C₁₅) radical, an unsubstituted or substituted heterocyclic radical, or a radical of the formula —NRₐ—X′—R_{b}, wherein X′ represents a valence bond or a divalent linking moiety selected from the group of —C(═O)—, —S(═O)₂— or —(CH₂)ₙ—, n being an integer from 1 to 6;
Rₐ represents hydrogen or an unsubstituted or substituted alkyl (C₁-C₆) radical;
R_{b} represents hydrogen, an unsubstituted or substituted alkyl (C₁-C₆) radical, an unsubstituted or substituted aryl (C₆-C₁₄) radical, an unsubstituted or substituted aralkyl (C₇-C₁₆) radical, an unsubstituted or substituted heterocyclic radical, an unsubstituted or substituted alicyclic (C₅-C₇) radical or a carbalkoxy radical;
R₁ represents an unsubstituted or substituted alkyl (C₁-C₆) radical;
said heterocyclic radical represented by R or R_{b} being at least one selected from the group consisting of furan, thiophene, pyrrole, tetrazole, pyridine, piperidine, morpholine, pyrazole, pyridazine, triazole, pyrimidine, oxadiazole, thiadiazole, oxazole, isoxazole, isothiazole, and azepane; said alkyl radical substituent(s) being at least one selected from the group consisting of carboxy, hydroxy, alkoxy, amino, alkylamino, dialkylamino, thiol and alkylthio; said aryl radical substituent(s) and said aralkyl radical substituent(s) being at least one selected from the group consisting of a straight or branched chain, saturated or unsaturated aliphatic group having 1-6 carbon atoms, halogen, nitro, carboxy, hydroxy, hydroxyalkyl, perhaloalkyl, monohaloalkyl, dihaloalkyl, alkoxy, perhaloalkoxy, phenylalkoxy, acyl, acyloxy, acyloxyalkyl, cyano, carbalkoxy, thiol, alkylthio, alkylsulfinyl, alkylsulfonyl, amino, alkylamino, dialkylamino, aminoalkyl, alkylaminoalkyl, dialkylaminoalkyl, sulfonamido, carboxamido, and alkanoylamino; said heterocyclic radical substituent(s) and said alicyclic radical substituents(s) being at least one selected from the group consisting of a straight or branched chain, saturated or unsaturated aliphatic group having 1-6 carbon atoms, halogen, perhaloalkyl, monohaloalkyl, dihaloalkyl, alkoxy, acyl, acyloxy, acyloxyalkyl, phenylalkoxy, hydroxy, hydroxyalkyl, alkylsulfonate, thiol, alkylthio, alkylsulfinyl, alkylsulfonyl, nitro, carboxy, carbalkoxy, or an unsubstituted and substituted aryl (C₆-C₁₄) radical;
R₂, R₃ and R₄ may be the same or different and represent hydrogen or a radical selected from the group consisting of substituted or unsubstituted straight or branched alkyl (C₁-C₆), substituted or unsubstituted alicyclic (C₅-C₇), substituted or unsubstituted aryl (C₆-C₁₄) radicals, or an amino acid residue and with the proviso that at least one of R₂, R₃, and R₄ must be other than hydrogen; and
the isomeric forms of said compound and the pharmaceutically acceptable salts of said compound.

5. A pharmaceutical composition for treating or preventing viral infections, said composition comprising a compound as claimed in claim 4 in an amount effective to attenuate viral infectivity, and a pharmaceutically acceptable carrier medium.

6. A pharmaceutical composition as claimed in claim 5, further comprising at least one supplemental active agent selected from the group of interferon, a pegylated interferon, ribavirin, protease inhibitors, polymerase inhibitors, small interfering RNA compounds, anti-sense compounds, nucleotide analogs, nucleoside analogs, immunoglobulins, immunomodulators, hepatoprotectants, anti-inflammatory agents, antibiotics, antivirals, and anti-infective compounds.

7. A compound for treating or preventing viral infection and disease associated with said infection in living hosts, said compound having the formula: 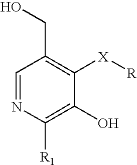
wherein X represents a divalent linking moiety selected from the group —CH═N— or —CH═CRₐ—;
R is a radical selected from the group consisting of an unsubstituted or substituted alkyl (C₁-C₆) radical, an unsubstituted or substituted aryl (C₆-C₁₄) radical, an unsubstituted or substituted aralkyl (C₇-C₁₅) radical, an unsubstituted or substituted heterocyclic radical, or a radical of the formula —NRₐ—X′—R_{b}, wherein X′ represents a valence bond or a divalent linking moiety selected from the group of —C(═O)—, —S(═O)₂— or —(CH₂)ₙ—, n being an integer from 1 to 6;
Rₐ represents hydrogen or an unsubstituted or substituted alkyl (C₁-C₆) radical;
R_{b} represents hydrogen, an unsubstituted or substituted alkyl (C₁-C₆) radical, an unsubstituted or substituted aryl (C₆-C₁₄) radical, an unsubstituted or substituted aralkyl (C₇-C₁₆) radical, an unsubstituted or substituted heterocyclic radical, an unsubstituted or substituted alicyclic (C₅-C₇) radical or a carbalkoxy radical;
R₁ represents an unsubstituted or substituted alkyl (C₁-C₆) radical; and
said heterocyclic radical represented by R or Rb being at least one selected from the group consisting of furan, thiophene, pyrrole, tetrazole, pyridine, piperidine, morpholine, pyrazole, pyridazine, triazole, pyrimidine, oxadiazole, thiadiazole, oxazole, isoxazole, isothiazole, and azepane; said alkyl radical substituent(s) being at least one selected from the group consisting of carboxy, hydroxy, alkoxy, amino, alkylamino, dialkylamino, thiol and alkylthio; said aryl radical substituent(s) and said aralkyl radical substituent(s) being at least one selected from the group consisting of a straight or branched chain, saturated or unsaturated aliphatic group having 1-6 carbon atoms, halogen, nitro, carboxy, hydroxy, hydroxyalkyl, perhaloalkyl, monohaloalkyl, dihaloalkyl, alkoxy, perhaloalkoxy, phenylalkoxy, acyl, acyloxy, acyloxyalkyl, cyano, carbalkoxy, thiol, alkylthio, alkylsulfinyl, alkylsulfonyl, amino, alkylamino, dialkylamino, aminoalkyl, alkylaminoalkyl, dialkylaminoalkyl, sulfonamido, carboxamido, and alkanoylamino; said heterocyclic radical substituent(s) and said alicyclic radical substituents(s) being at least one selected from the group consisting of a straight or branched chain, saturated or unsaturated aliphatic group having 1-6 carbon atoms, halogen, perhaloalkyl, monohaloalkyl, dihaloalkyl, alkoxy, acyl, acyloxy, acyloxyalkyl, phenylalkoxy, hydroxy, hydroxyalkyl, alkylsulfonate, thiol, alkylthio, alkylsulfinyl, alkylsulfonyl, nitro, carboxy, carbalkoxy, or an unsubstituted and substituted aryl (C₆-C₁₄) radical; the isomeric forms of said compound and the pharmaceutically acceptable salts of said compound.

8. A pharmaceutical composition for treating or preventing viral infections, said composition comprising a compound as claimed in claim 7 in an amount effective to attenuate viral infectivity, and a pharmaceutically acceptable carrier medium.

9. A pharmaceutical composition as claimed in claim 8 further comprising at least one supplemental active agent selected from the group of interferon, a pegylated interferon, ribavirin, protease inhibitors, polymerase inhibitors, small interfering RNA compounds, anti-sense compounds, nucleotide analogs, nucleoside analogs, immunoglobulins, immunomodulators, hepatoprotectants, anti-inflammatory agents, antibiotics, antivirals, and anti-infective compounds.

10. A method of treating or preventing infections caused by viruses of the Flaviviridae family and diseases associated with said infections in a living host having or susceptible to said infections, said method comprising administering to said living host a therapeutically effective amount of at least one compound, including the pharmaceutically acceptable salts thereof, selected from the group consisting of compounds of the formula: 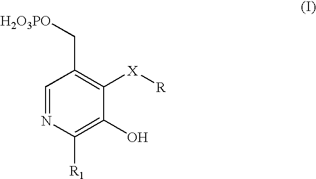
wherein X represents a divalent linking moiety selected from the group —CH═N— or —CH═CRₐ—;
R is a radical selected from the group consisting of an unsubstituted or substituted alkyl (C₁-C₆) radical, an unsubstituted or substituted aryl (C₆-C₁₄) radical, an unsubstituted or substituted aralkyl (C₇-C₁₅) radical, an unsubstituted or substituted heterocyclic radical, or a radical of the formula —NRₐ—X′—R_{b}, wherein X′ represents a valence bond or a divalent linking moiety selected from the group of —C(═O)—, —S(═O)₂— or —(CH₂)ₙ—, n being an integer from 1 to 6;
Rₐ represents hydrogen or an unsubstituted or substituted alkyl (C₁-C₆) radical;
R_{b} represents hydrogen, an unsubstituted or substituted alkyl (C₁-C₆) radical, an unsubstituted or substituted aryl (C₆-C₁₄) radical, an unsubstituted or substituted aralkyl (C₇-C₁₆) radical, an unsubstituted or substituted heterocyclic radical, an unsubstituted or substituted alicyclic (C₅-C₇) radical or a carbalkoxy radical;
R₁ represents an unsubstituted or substituted alkyl (C₁-C₆) radical;
said heterocyclic radical represented by R or R_{b} being at least one selected from the group consisting of furan, thiophene, pyrrole, tetrazole, pyridine, piperidine, morpholine, pyrazole, pyridazine, triazole, pyrimidine, oxadiazole, thiadiazole, oxazole, isoxazole, isothiazole, and azepane; said alkyl radical substituent(s) being at least one selected from the group consisting of carboxy, hydroxy, alkoxy, amino, alkylamino, dialkylamino, thiol and alkylthio; said aryl radical substituent(s) and said aralkyl radical substituent(s) being at least one selected from the group consisting of a straight or branched chain, saturated or unsaturated aliphatic group having 1-6 carbon atoms, halogen, nitro, carboxy, hydroxy, hydroxyalkyl, perhaloalkyl, monohaloalkyl, dihaloalkyl, alkoxy, perhaloalkoxy, phenylalkoxy, acyl, acyloxy, acyloxyalkyl, cyano, carbalkoxy, thiol, alkylthio, alkylsulfinyl, alkylsulfonyl, amino, alkylamino, dialkylamino, aminoalkyl, alkylaminoalkyl, dialkylaminoalkyl, sulfonamido, carboxamido, and alkanoylamino; said heterocyclic radical substituent(s) and said alicyclic radical substituents(s) being at least one selected from the group consisting of a straight or branched chain, saturated or unsaturated aliphatic group having 1-6 carbon atoms, halogen, perhaloalkyl, monohaloalkyl, dihaloalkyl, alkoxy, acyl, acyloxy, acyloxyalkyl, phenylalkoxy, hydroxy, hydroxyalkyl, alkylsulfonate, thiol, alkylthio, alkylsulfinyl, alkylsulfonyl, nitro, carboxy, carbalkoxy, or an unsubstituted and substituted aryl (C₆-C₁₄) radical; and the isomeric forms of said compound;
a compound of the formula: 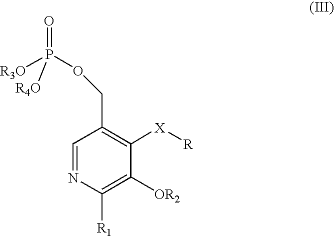
wherein X, R, and R₁ are as defined above;
R₂, R₃ and R₄ may be the same or different and represent hydrogen or a radical selected from the group consisting of substituted or unsubstituted straight or branched alkyl (C₁-C₆), substituted or unsubstituted alicyclic (C₅-C₇), substituted or unsubstituted aryl (C₆-C₁₄) radicals, or an amino acid residue and with the proviso that at least one of R₂, R₃, and R₄ must be other than hydrogen;
the isomeric forms of said compound and the pharmaceutically acceptable salts of said compound; and
a compound of the formula: 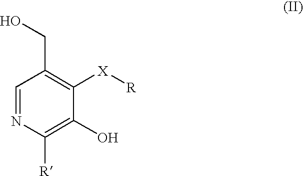
wherein X, R, and R₁ are as defined above, and the isomeric forms of said compound or a precursor of said compound.

11. A method as claimed in claim 10, wherein said compound or a precursor of said compound is administered to a living host in unit dosage form containing from about 0.001 to about 120 mg of said compound per kilogram of body weight per day, said unit dosage optionally including a pharmaceutically acceptable carrier medium.

12. A method as claimed in claim 10, wherein a precursor of said compound is administered in the form of a prodrug.

13. A method as claimed in claim 10, wherein said at least one compound or precursor of said compound is administered in combination, either concurrently or sequentially, with at least one other biologically active agent.

14. A method as claimed in claim 13, wherein said other biologically active agent is selected from the group of interferon, a pegylated interferon, ribavirin, protease inhibitors, polymerase inhibitors, small interfering RNA compounds, anti-sense compounds, nucleotide analogs, nucleoside analogs, immunoglobulins, immunomodulators, hepatoprotectants, anti-inflammatory agents, antibiotics, antivirals, and anti-infective compounds.

15. A method as claimed in claim 10, wherein at least two different compounds selected from Formula I, Formula II and Formula III, or a precursor thereof, are administered in combination, either concurrently or sequentially.

16. A method as claimed in claim 15 which further comprises administering at least one additional therapeutic agent or potentiator, selected from the group consisting of acyclovir famicyclovir, valgancyclovir, ribavirin, amantadine, an interferon or a derivative of said therapeutic agents or potentiators.

17. A method as claimed in claim 16, wherein said additional therapeutic agent or potentiator, or derivative thereof, is administered concurrently with said at least two different compounds selected from Formula I, Formula II and Formula III, or a precursor thereof.

18. A method as claimed in claim 17, wherein said at least two different compounds selected from Formula I, Formula II and Formula III, or a precursor thereof, and said at least one additional therapeutic agent or potentiator are administered in a single dose form.

19. A method as claimed in claim 17, wherein one or more of said at least two different compounds selected from Formula I, Formula II and Formula III, or a precursor thereof, and said additional therapeutic agent or potentiator are administered in separate dosage forms.

20. A method as claimed in claim 16, wherein said additional therapeutic agent or potentiator, or derivative thereof, is administered sequentially with said at least two different compounds selected from Formula I, Formula II and Formula III, or a precursor thereof.

21. A method as claimed in claim 16, wherein said therapeutic agent or potentiator is selected from the group consisting of interferon alpha-2a, interferon alpha-2b or a polyethylene glycol-modified conjugate of interferon alpha-2a or interferon alpha-2b.

22. A method as claimed in claim 21, wherein said therapeutic agent or potentiator is interferon alpha-2b or a polyethylene glycol-modified conjugate thereof.

23. A method as claimed in claim 15 which further comprises administering at least one inhibitor of the HCV life cycle selected from the group consisting of inhibitors of HCV cell attachment or virus entry, inhibitors of HCV translation, inhibitors of HCV RNA transcription or replication, inhibitors of HCV maturation, inhibitors of assembly or virus release or inhibitors of HCV enzyme activities.

24. A method as claimed in claim 23, wherein said inhibitors of HCV enzyme activities are selected from the group consisting of inhibitors of HCV nucleotidyl transfrase, HCV helicase, HCV protease or HCV polymerase.

25. A method as claimed in claim 10, wherein said at least one compound or a precursor of said compound is administered orally.

26. A method as claimed in claim 10, wherein said at least one compound or a precursor of said compound is administered rectally.

27. A method as claimed in claim 10, wherein said at least one compound or a precursor of said compound is administered parenterally.

28. A method as claimed in claim 10, wherein said at least one compound or a precursor of said compound is administered intracisternally.

29. A method as claimed in claim 10, wherein said at least one compound or a precursor of said compound is administered intravaginally.

30. A method as claimed in claim 10, wherein said at least one compound or a precursor of said compound is administered intraperitoneally.

31. A method as claimed in claim 10, wherein said at least one compound or a precursor of said compound is administered locally.

32. A method as claimed in claim 10, wherein said at least one compound or a precursor of said compound is administered by inhalation.

33. A method as claimed in claim 10, wherein said viruses of the Flaviviridae family are selected from the group consisting of viruses of the hepacivirus genus, viruses of the pestivirus genus, viruses of the flavivirus genus and viruses unassigned to particular genera within the Flaviviridae family.

34. A method as claimed in claim 33, wherein said at least one compound or a precursor of said compound is administered to living hosts in unit dosage form containing about 0.001 to about 120 mg of said compound per kilogram of body weight per day.

35. A method as claimed in claim 33, wherein a precursor of said compound is administered in the form of a prodrug.
